(19) United States
(12) Patent Application Publication
(10) Pub. No.: US 2008/0248058 A1
(43) Pub. Date: Oct. 9, 2008

(54) Plant Disease Control Agent And Method For Controlling Plant Disease

(75) Inventors: Kiyohiko Nakasaki, Shizuoka (JP); Miyuki Saito, Okazaki-shi Aichi (JP)

Correspondence Address: MARSHALL, GERSTEIN & BORUN LLP, 233 S. WACKER DRIVE, SUITE 6300, SEARS TOWER, CHICAGO, IL, 60606, US

(73) Assignee: National University Corporation Shizouka University, Shizouka (JP)

(21) Appl. No.: 11/815,743

(22) PCT Filed: Feb. 8, 2006
(86) PCT No.: PCT/JP2006/302197

§ 371 Date: Mar. 17, 2008

(30) Foreign Application Priority Data
Feb. 8, 2005 (JP) 2005-031600
Mar. 31, 2005 (JP) 2005-104678

(51) Int. Cl. A01N 65/00; A01H 15/00; A01P 3/00

(52) U.S. Cl. 424/195.15; 800/297

(57) ABSTRACT

The present invention provides a plant disease control agent which is stable and safe in terms of leaving no residue and a method for controlling plant diseases using the same. The plant disease control agent comprises a fragment of an inky cap mushroom, and plant diseases are controlled by using the plant disease control agent. The plant disease control agent may be a suspension comprising the fragment of the inky cap mushroom or a solid material composed of the suspension and a carrier to which the suspension is absorbed. The inky cap mushroom is preferably Coprinus curtus or the like, especially preferably Coprinus curtus GM-21 (NITE BP-37).

### TECHNICAL FIELD

The present invention relates to a plant disease control agent and a method for controlling plant disease, and in particular, to a plant disease control agent and a method for controlling plant disease by using an organism.

### BACKGROUND ART

Fungi (filamentous fungi), or molds, induce diseases such as damping off, root rot, leaf rot, and wilt in many vegetables such as cabbage, cucumber, tomato, eggplant and rape leaf, agricultural products such as rice, flowers, trees, turfs, and others. Well-known pathogenic fungi include strains belonging to the genus Rhizoctonia, the genus Fusarium, the genus Pytium, the genus Trichoderma, the genus Sclerotium, and the like. Although, chemicals, so-called agricultural chemicals, are generally applied in order to prevent such fungal plant diseases, there is recently concern about the residues therefrom. Thus, a disease controlling technique that is safe for the environment is desired.

From this recent background, biological control methods with microbes (so-called biological pesticides), which would be safer to the environment, have been proposed, and some of these have already been put into practice. For example, there is a method of controlling plant diseases caused by fungi using a bacterium belonging to the genus Pseudomonas disclosed (see Japanese Patent Application Laid-open (JP-A) No. H11-87866). However, the control effects of the bacterium belonging to the genus Pseudomonas are considered to be derived from the antimicrobial substances produced by the bacterium, and thus, there is a concern about its safety when used in large amounts.

On the other hand, a disease control method using a bacterium belonging to the genus Bacillus has also been disclosed (see New Soil Microbes (2), Plant growth and microorganism, Soil Microorganism Research Society Ed., Hakuyusha p. 129-131). However, the control effect of the bacteria belonging to the genus Pseudomonas or the genus Bacillus against fungal plant diseases are not able to demonstrate such a stable effect as may be achieved with chemicals, and they have the disadvantage that their effects are readily destabilized depending on, for example, the soil conditions. That is, in specific environments, pathogenic fungi often proliferate predominantly, and control by the bacteria does not work sufficiently effectively.

Also disclosed is a method of using a non-pathogenic Trichoderma or Mucor sp. fungus (see JP-A No. H10-150978) and a method of using a fungus belonging to a non-pathogenic the genus Fusarium fungus (see Japanese Patent Application No. H9-530003). In both these methods disease is controlled by antagonism between the non-pathogenic fungus used and the pathogenic fungus, however, there have been some cases in which the effect is not sufficiently exhibited due to the fungus not growing sufficiently in soil or the like.

### DISCLOSURE OF THE INVENTION

### Problems for Solving the Invention

Accordingly, an object of the present invention is to provide a control agent for controlling plant disease which is stable and safe in terms of leaving no residue.

### Means to Solve the Problems

The plant disease control agent according to the invention is characterized by comprising fragment(s) of an inky cap mushroom.

The plant disease control agent may be a suspension containing the fragment of inky cap mushroom or a solid material composed of the suspension and a carrier to which the suspension is absorbed.

The inky cap mushroom is preferably a strain belonging to the genus Coprinus or the genus Psathyrella, more preferably at least one selected from the group consisting of Coprinus curtus, Coprinus cinereus, Coprinus disseminatus, Coprinus comatus, Coprinus atramentarius, Coprinus radians, Psathyrella multissima, Psathyrella candolleana and Psathyrella velutina, and particularly preferably Coprinus curtus GM-21 (NITE BP-37).

The method for controlling plant disease according to the invention is characterized by controlling plant diseases by using the plant disease control agent.

The pathogenic microbe of the plant disease may be a fungus, and the plant disease fungus may be a fungus of the genus Rhizoctonia or the genus Fusarium.

### EFFECT OF THE INVENTION

According to the invention, a plant disease can controlled stably and safely in terms of leaving no residue.

### BRIEF DESCRIPTION OF THE DRAWINGS

FIG. 1 is a chart showing the confirmed control effect on the pathological state of Pak-choi bottom rot in Example 1 of the present invention.

FIG. 2 is a chart showing the confirmed control effect on the pathological state of Rhizoctonia patch disease in mascarenegrass in Example 2 of the invention.

FIG. 3 is a chart showing the confirmed control effect on the pathological state of Rhizoctonia patch disease in mascarenegrass in Example 3 of the invention.

FIG. 4 is a chart showing the confirmed control effect on the pathological state of Pak-choi bottom rot in Example 4 of the invention.

FIG. 5 is a chart showing the confirmed control effect on the pathological state of Pak-choi bottom rot in Example 5 of the invention.

FIG. 6 is a chart showing the confirmed control effect on the pathological state of lettuce bottom rot in Example 7 of the invention.

### BEST MODE OF CARRYING OUT THE INVENTION

The plant disease control agent according to the present invention contains a fragment of an inky cap mushroom.

The inventors considered that it would be advantageous to use a Eumycetes as a controlling agent for pathogenic fungi since pathogenic fungi are Eumycetes, and, by screening fungi isolated from soil for a control effect, it was found that an inky cap mushroom, a Basidiomycetes mushroom, which is one of the more developed Eumycetes, had a remarkable control effect.

The inky cap mushroom is a common mushroom that grows naturally in fields. The inky cap mushroom for use in the invention belongs to the family Coprinaceae, and, from the viewpoint of safety, it preferably belongs to the genus Coprinus or the genus Psathyrella, that are not the genus Panaeolus that is a so-called poisonous mushroom. Among them, Coprinus curtus, Coprinus cinereus, Coprinus disseminatus, Coprinus comatus, Coprinus atramentarius, Coprinus radians, Psathyrella multissima, Psathyrella candolleana, and Psathyrella velutina are preferably, and these may be used singly or in combinations thereof.

Among these, a novel isolated strain GM-21 belonging to Coprinus curtus is particularly preferable, because it is particularly effective in controlling plant diseases, The Coprinus curtus GM-21 is a strain isolated in screening study under the hypothesis that there could be obtained some Eumycetes that act against pathogenic fungi in the soil from near the roots of healthy vegetables. More specifically, the roots of healthy growing Pak-choi were placed in a suspension together with the surrounding soil in sterile water; the suspension was spread onto a PDA culture medium; and all fungi grown thereon were isolated. By using each of the isolated fungi, Pak-choi was grown in a pot containing pathogenic microbe Pak-choi 2 causing Pak-choi bottom rot, and the pathogen-controlling efficiency thereof was studied. The strain having remarkable effect was isolated as GM-21. GM-21 was deposited with the International Patent Organism Depository as NITE P-37 on Nov. 18, 2004 (international deposit number: NITE BP-37).

The taxonomic properties of the Coprinus curtus CM-21 are as follows:

### (1) Culture Plate Appearance

Macroscopic observation (25° C.) of an agar plate of potato dextrose medium (PDA: Eiken Chemical Co., Ltd., pH 5.6) showed a white colony (1A-1: color code number used in Kornerup A. and Wanscher, J. H. (1978) Methuen handbook of color, 3rd ed., Eyre Methuen, London, UK, pp, 243), with wool-like surface state and having a diameter of approximately 80 mm. No soluble dye was detected.

### (2) Growth Temperature Test

The microbe showed good growth at a temperature in the range of 20° C. to 40° C. and most rapidly at 25° C. to 27° C. The colony diameter after culture for one week under various temperature conditions was as follows: 20° C., 60 to 62 mm; 23° C., 78 to 80 mm; 22° C., 80 to 81 mm; 27° C., 82 to 84 mm; 30° C., 75 to 80 mm; 37° C., 70 to 75 mm; and 40° C., 63 to 65 mm.

### (3) Microscopic Observation

The cap of the fruit body had a diameter of 2 to 10 mm, was white, gray or yellow gray, and had a cotton-like surface and radial deep gills could be seen. The hymenium surface was covered with gills, and the spores were white when immature, and changed from black to black brown gradually with maturation of the gills and accompanying liquefaction of the gill portion. There were spherical to semi-spherical cells observed on the cap surface with slightly wart-like surfaces. The basidium was rod-shaped with a one chamber without a barrier wall and having a projection called sterigma on the top, from which a basidiospore was observed to be formed.

The basidiospore was an elliptic cell having a diameter of 8 to 10×5 to 7 μm that was brown to dark brown in color when mature; the surface was smooth; there was a germ pore (darkened area) in a one end; and a projection that had been bound to the sterigma was sticking outward in the other end.

### (4) Molecular Phylogeny Analysis

ITS-5.8S rDNA nucleotide sequence data of GM-21 is shown in Table 1 (SEQ ID No. 1).

TABLE 1

| | |
|---|---|
| tttccgtagg tgaacctgcg gaaggatcat taacgaataa ctatggtgtt ggttgtagct | 60 |
| gcctcctcgg aggaatgtgc acgcccgcca tttttatctt tccacctgtg caccgactgt | 120 |
| aggtctggat aactctcgcc tcacggcaga tgcgaggatt ggcctctgtg cctctcctcg | 180 |
| aatttccagg ctctacgtct tttacacacc ccaatagtat gatatagaat gtagtcaatg | 240 |
| ggcttcttag cctataaaac actatacaac tttcagcaac ggatctcttg gctctcgcat | 300 |
| cgatgaagaa cgcagcgaaa tgcgataagt aatgtgaatt gcagaattca gtgaatcatc | 360 |
| gaatctttga acgcaccttg cgctccttgg tattccgagg agcatgcctg tttgagtgtc | 420 |
| attaaattct caacctcgcc agttttctga actgcgtcga ggcttggatt gtgggggttt | 480 |
| gtgcaggctg cctcagcgtg gtctgctccc ctgaaatgca ttagcgagtt catactgagc | 540 |
| tccgtctatc ggtgtgataa ttatctacgc cgttagtcga gttcagactt gcttctaacc | 600 |
| gtccgcaagg acaactcttg acaatttgac ctcaaatcag gtaggactac ccgctgaact | 660 |
| taa | 663 |

On the basis of the ITS-5.8S rDNA nucleotide sequence data, BLAST homology search and molecular phylogeny analysis on nucleotides including those in relevant classification groups were performed with reference to international DNA nucleotide sequence databases, and the results are summarized in Table 2. From the characteristics of the nucleotide sequence, GM-21 was found to be a new species of fungus, Coprinus curtus Kalchbr. ex Thum., on the basis of Kirk, P. M. Cannon, P. F. David J. C. and Stalpers, J. A. (2001) Ainsworth & Bisby's Dictionary of the Fungi, CAB International, Wallingford, UK, p. 655.

TABLE 2

| | | Accession | |
|---|---|---|---|
| Entry name | Strain name | No | Identity |
| Coprinellus curtus | | AY461824 | 642/643 = 99.8% |
| Coprinellus curtus | | AY461834 | 607/611 = 99.3% |
| Coprinellus | KACC49407 | AF361228 | 601/653 = 92.0% |
| xanthothrix | | | |
| Coprinellus | KACC49382 | AF345818 | 472/500 = 94.4% |
| flocculosus | | | |
| Coprinellus | | AY461838 | 572/632 = 90.5% |
| disseminatus | | | |
| Coprinellus radians | | AY461815 | 442/467 = 94.6% |
| Coprinellus | | AY461832 | 449/482 = 93.2% |
| micaceus | | | |
| Coprinopsis | KACC49375 | AF345813 | 449/494 = 90.9% |
| lagopus | | | |
| Coprinus | | AY461814 | 389/427 = 91.1% |
| cordisporus | | | |
| Coprinellus | KACC49409 | AF345824 | 371/413 = 89.8% |
| bisporus | | | |
| Psathyrella | KACC500091 | AF345810 | 254/268 = 94.8% |
| candolleana | | | |
| Lepiota | | AY176386 | 186/186 = 100.0% |
| ochraceofulva | | | |
| Lacrymaria | KACC500079 | AF345811 | 242/258 = 93.8% |
| velutina | | | |
| Leucoagaricus | | AY176407 | 203/210 = 96.7% |
| americanus | | | |
| Leucoagaricus | | AF295931 | 203/210 = 96.7% |
| barssii | | | |
| Leucoagaricus | | AY243631 | 203/210 = 96.7% |
| cupresseus | | | |
| Macrolepiota | | U85310 | 216/226 = 95.6% |
| procera | | | |
| Macrolepiota velosa | | AF482853 | 219/230 = 95.2% |
| Apterostigma | | AF079674 | 211/221 = 95.5% |
| auriculatum | | | |
| symbiont S50 | | | |
| Cyphomyrmex | | AF079679 | 211/221 = 95.5% |
| longiscapus | | | |
| symbiont S36 | | | |

The inky cap mushroom used for the plant disease control agent according to the invention may be cultivated or propagated. The cultivation or propagation is performed by a traditionally known method, for example, by cultivating or propagating the fragment of a mycelium, a spore or a fruit body of the inky cap mushroom using an agar plate culture or a liquid medium culture. There is no particular limitation to the kinds of the medium used, but potato dextrose (hereinafter “PD”) medium, Czapek Dox medium, and the like may be favorably used; and cultivation can be performed under aerobic conditions, for example, by agar plate culture, shaking culture, aerated culture, or the like. The cultivation temperature is, for example, 20 to 40° C., preferably 25 to 27° C.; the pH is between 5 to 8; the cultivation period is suitably about 1 to 20 days, and more preferably about 4 to 14 days from the viewpoint of efficiency.

After growing in culture, the cultured strain may be used either in the form of mycelium (hypha), spore, or fruit body. The strain may be reduced to fragments with a suitable size, for example, by using a cutting blade either as it is or after drying, depending on the strain. When the mycelium is divided in a homogenizer, the fragments of mycelium generally have a diameter of approximately 3 mm at the most, and mostly are of a diameter less than that. The spores of the strain can be used as a fragment having the size as they are. The fruit body of the strain can be, for example, cut into pieces of 1 mm square. Although any size, that is, larger or smaller than that described above, of the fragment is acceptable, reduction in size is favorable for dispersion in or absorption onto something.

In the invention, as long as the plant disease control agent contains the fragment of an inky cap mushroom, there is no limitation to the manner in which it is contained, and it may be a suspension containing fragment of an inky cap mushroom. In preparing a suspension by homogenization of the inky cap mushroom fragment in a liquid, preparation is readily made using a homogenizer equipped with an agitating blade. When doing so, sterile water and the like may be used as the liquid to disperse the strain, and saline solution and phosphate salts can be added in order to stabilize the strain.

The suspension concentration is preferably 0.01 to 10 parts by weight, more preferably 0.1 to 5 parts by weight, in terms of strain dry weight with respect to 100 parts by weight of water. A concentration higher that the above is unfavorable, because the strain may be less easily dispersed and even if it is absorbed to a mineral, soil or compost, or spread directly on the soil, it may become difficult to blend uniformly. On the other hand, a concentration less than the above is also unfavorable, because a suspension thereof is too dilute with water, and even when absorbed on something it is inefficient to use in practice. Of course the obtained suspension may be used as it is, or concentrated or diluted as required.

In using such a suspension, it is preferable to make a solid material composed of the suspension and a carrier in which the suspension is absorbed, in order to improve the convenience of handling and storage stability. The carrier for use is preferably a carrier capable of holding the strain uniformly, and favorable examples thereof include: porous particulate materials such as pearlite, vermiculite, zeolite, diatomaceous earth, and Kanuma soil (a weathered pumice) and the like; mineral powders such as talc, clay, and calcium carbonate and the like; polymer compounds such as polyvinylalcohol and the like; natural polymer compounds such as xanthan gum and alginic acid and the like; and the like. When the suspension is absorbed to a compost, the kind of the compost used is not particularly limited, but well matured compost, for example completely matured compost, is preferable. Food waste such as soy bean cake may also be used.

Before the suspension is absorbed on a carrier, a protecting agent may be added to the suspension. The protecting agent for use is, for example, a sugar such as glucose, fructose, sucrose or trehalose, or a mixture of plural thereof, or a protein, or the like. When using a protecting agent, the protecting agent may be added in an amount similar to that commonly practiced in the art.

Absorption on carrier may be carried out easily according to a common method, for example, by mixing a carrier with a suspension and drying the mixture. Operations such as drying are performed under conditions in which the strain is not killed,

The plant disease control agent according to the invention is not limited to a solid material containing the suspension and the carrier, and any kind of preparation such as a wettable powder, an emulsion, an oil, granules, a powder, tablets, or capsules may be used, as long as the strain is kept alive. Alternatively, a suspension itself or a mixture with another solution may be used as a seed-coating agent to be applied on the seed surface. Other additives and processing conditions needed for producing such preparations are known to and selected easily by those who are skilled in the art.

The method for controlling plant disease according to the invention employs the plant disease control agent described above.

The plant disease control agent is used in various applications according to the kind of the preparation. For example, it may be spread directly around the roots of plants as a suspension or a wettable powder, or alternatively, may be used blended with another solution, a soil such as a culture medium or potting sol, or a culture solution such as that of a hydroponic solution for hydroponic cultivation. When spraying or using as a blend it is effective and favorable to apply around the roots of plants. The blending amount thereof applied to the soil or culture medium varies according to the relative conditions such as concentration of the pathogenic microbe and the like, but is preferably an amount of inky cap mushroom that is 2 ppm or more, more preferably 40 ppm or more. When the plant disease control agent is used as a seed-coating agent, it may be applied in a suitable amount to the surface of the seeds before sowing, and the coated seeds may be sowed in that state.

The plant disease control agent according to the invention has control effect against plant diseases, but when used for plant diseases in which the pathogenic microbe is a fungus, improved controlling effectiveness is demonstrated and so this is preferable. The controlling effect is particularly significant, when it is used against plant diseases caused by fungus belonging to the genus Rhizoctonia and the genus Fusarium. The plant diseases possibly treated with the plant disease control agent according to the invention include Pak-choi bottom rot, turf leaf rot, lettuce bottom rot, melon wilt, tomato wilt, and the like.

It is possible to control plant diseases stably and safely in terms of leaving no residue by using the plant disease control agent according to the invention.

Hereinafter, the invention will be described specifically with reference to Examples, but it should be understood that the invention is not restricted by these

### EXAMPLES

### Example 1

Coprinus curtus Kalchbr. ex Thum. GM-21 was inoculated in a PD liquid medium, cultured stationarily in an incubator at 27° C. for about 5 days, and filtered through sterilized gauze to obtain a required amount. Five ml of sterile water was added with respect to 1 g of hypha thereof, and the mixture obtained was homogenized, to give a suspension A, as a plant disease control agent according to the invention.

As Comparative Examples, a suspension B was prepared with Mucor sp. and a suspension C with Penicillium sp., respectively, similarly to the suspension A.

The control effect of the suspension A to C against a pathogenic microbe of Pak-choi bottom rot, Rhizoctonia solani Pak-choi 2, was determined in the following manners:

50 ml of sterile water was added onto a PD agar medium having Rhizoctonia solani Pak-choi 2 grown thereon; the mixture was homogenized to give a pathogenic fungal suspension; and the controlling effect of the suspension A to C against the bottom rot of Pak-choi was determined.

Specifically, 50 g of cultivation soil was placed in a plant test pot (manufactured by Asahi Techno Glass Corporation) and sterilized in an autoclave; 2 ml of the pathogenic microbe suspension, 1 ml of the suspension A, and 10 ml of sterile water was added thereto; and after the mixture was agitated, 20 surface sterilized Pak-choi seeds were added thereto. The pot was left in a plant growth chamber (manufactured by Shimadzu Rika Corporation) under the conditions of 39° C. in the daytime and 20° C. at night and a humidity of 55% for one month, for observation of disease development. The disease rate was calculated as follows:

Disease incidence (%)=(1a+2b−3c+4d+5e)×100/(5×seed number)
a: Number of individuals slightly infected
b: Number of individuals infected
c: Number of individuals significantly infected
d: Number of individuals withered
e: Number of individuals not geminated or withered to death

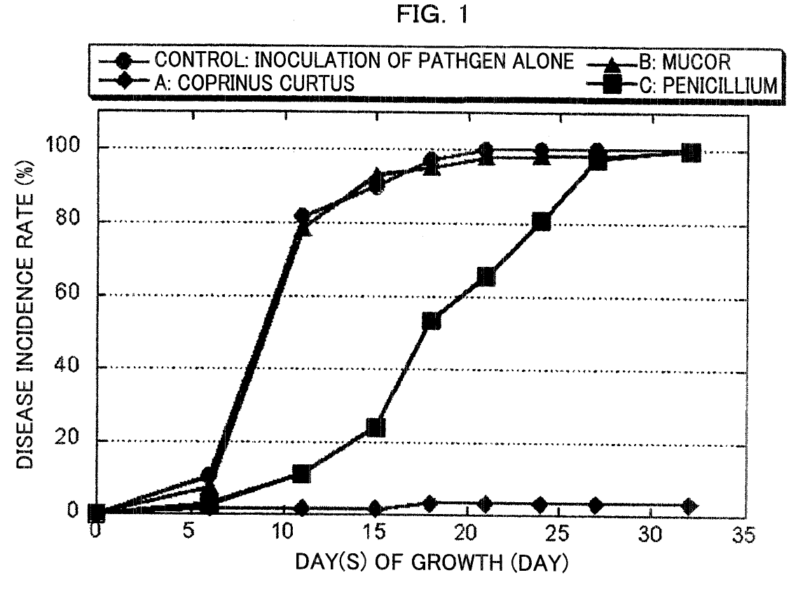

The results of the test on disease development, which is expressed by disease incidence rate, are shown in FIG. 1.

As apparent from FIG. 1, the suspension A using Coprinus curtus GM-21 bad a remarkable disease-controlling effect, suppressing development of the disease over 30 days or more. In contrast, although the suspension C using Penicillium could control the development during the early and intermediate periods of growth, it did not have a control effect as high as that of the suspension A, and lost most of the disease-controlling effect in the later period of growth, especially after 25 days. Use of a Penicillium sp. is also disadvantageous in that it also weakens the activity of the plant itself. On the other hand, the suspension B using Mucor did not show any disease control effect.

### Example 2

The effect of the suspension A to C against a pathogenic microbe of turf leaf rot (Rhizoctonia patch disease in mascarenegrass), Rhizoctonia solani K1, was determined in the following manner:

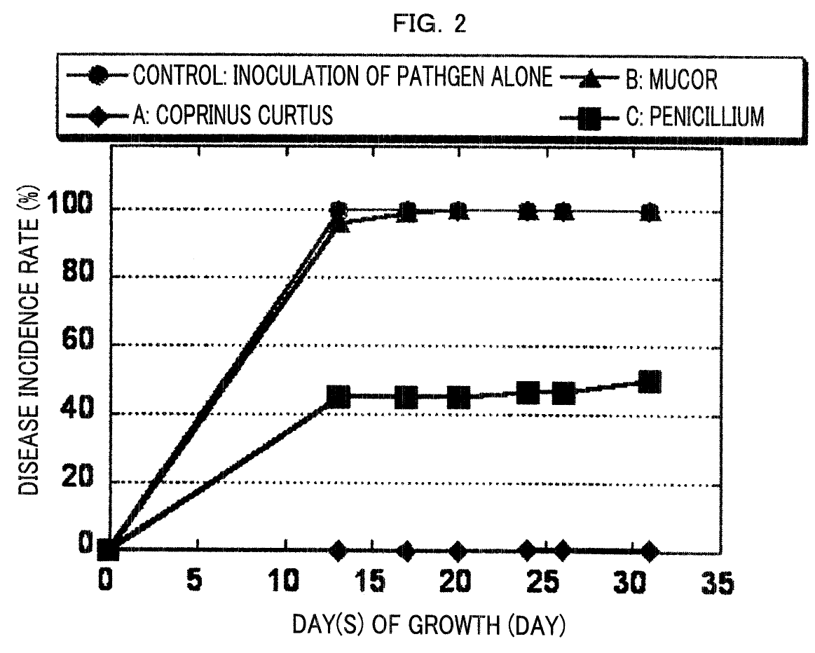

50 ml of sterile water was added on to an agar plate containing PD agar medium dish having Rhizoctonia solani K1 grown thereon; the mixture was homogenized and diluted 1000 fold with sterile water, to give a pathogenic microbe suspension; the each of the above suspension A, B, and C were mixed therewith; and the control effect of the mixture on Rhizoctonia patch disease in mascarenegrass was evaluated. Specifically, similarly to Example 1, 12 ml of the pathogenic microbe suspension and 1.5 ml each of respective suspensions were placed in a sterilized pot containing cultivation soil separately prepared and mixed thoroughly therein; 20 surface sterilized grass seeds were added thereto; and the disease development was observed in a plant growth chamber under a condition similar to that in Example 1. The results are summarized in FIG. 2.

As apparent from FIG. 2, the disease-controlling efficiency by the suspension A was also significant to this pathogenic microbe. In contrast, although the suspension C could control the development of the disease in the later phase of growth, it could not control the development in the early phase of growth, while the suspension B could not control the development at all.

### Example 3

In addition to the suspension A, a suspension D containing Coprinus cinereus NBRC30114 and a suspension E containing Coprinus disseminatus NBRC30972 were prepared respectively, in a similar manner to the suspension A.

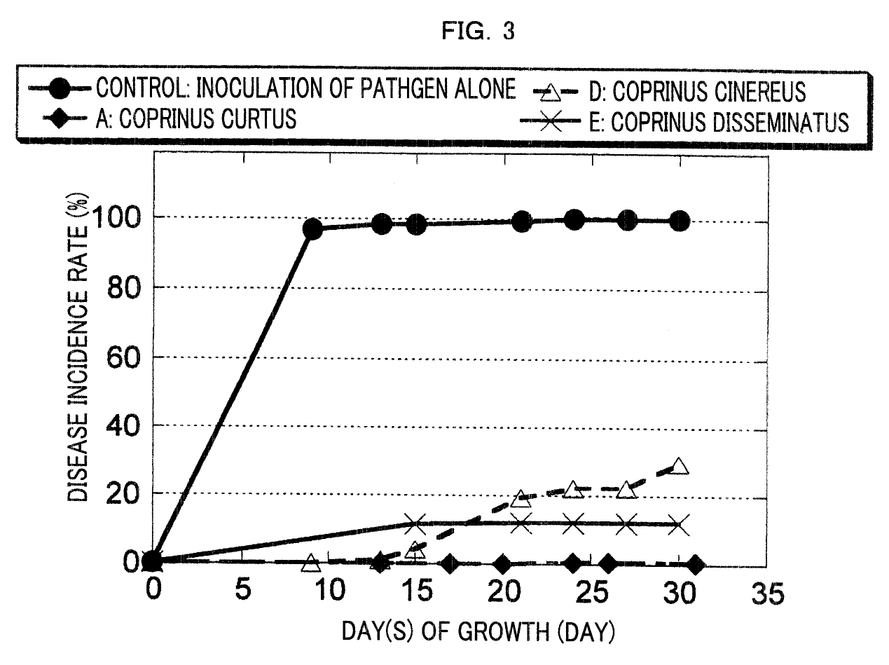

The control effect of the suspension A, D, and F on Rhizoctonia patch disease in mascarenegrass by Rhizoctonia solani K1 was evaluated in a similar manner to Example 2, except that 2 ml of the pathogenic microbe suspension, 2 ml of each suspension, and 8 ml of sterile water were used. The results are summarized in FIG. 3.

As apparent from. FIG. 3, the suspension D and E also suppressed the development in the growing period of 30 days, similarly to the suspension A. The suspension D controlled the development in stable manner, especially in the early phase of growth, while the suspension E in stable manner, especially in the middle phase of growth.

### Example 4

Coprinus curtus Kalchbr. Ex Thum. GM-21 was inoculated to PD liquid medium and cultured stationarily in an incubator at 27° C. for 13 days, and the mixture was filtered through sterilized gauze, to obtain a required amount. Fifty ml of sterile water was added with respect to 1 g of hypha, and the mixture obtained was homogenized, to give a suspension A2.

Coprinus comatus was inoculated to PD liquid medium and cultured stationarily in an incubator at 27° C. for 17 days, and the mixture was filtered through sterilized gauze, to obtain a required amount. Fifty ml of sterile water was added with respect 1 g of hypha, and the mixture obtained was homogenized, to give a suspension F.

Psathyrella velutina was inoculated in PD liquid medium and cultured stationarily in an incubator at 27° C. for 17 days, and the mixture was filtered through sterilized gauze, to obtain a required amount. Fifty ml of sterile water was added with respect to 1 g of hypha, and the mixture obtained was homogenized, to give a suspension G.

The effect of the suspension A2, G, and F against a pathogenic microbe of Pak-choi bottom rot, Rhizoctonia solani Pak-choi 2, was determined in the following manner:

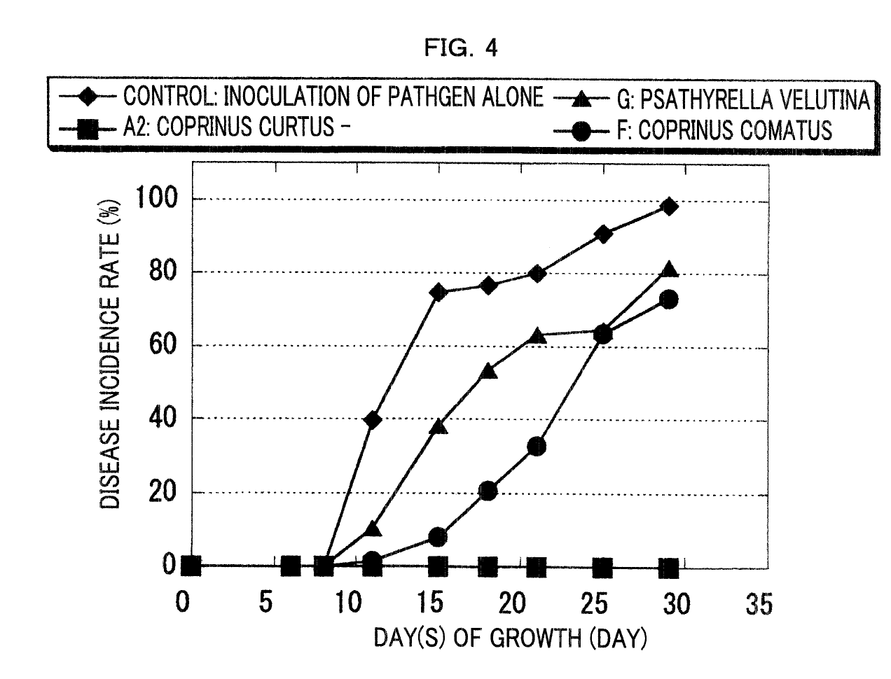

Fifty ml of sterile water was added onto a dish containing PDA agar culture medium dish having Rhizoctonia solani K1 grown thereon; the mixture was homogenized and diluted 10 fold with sterile water, to give a pathogenic microbe suspension; the control effects of the Coprinus curtus GM-21 suspension A2, the Coprinus comatus suspension F, and the Psathyrella velutina suspension C on Pak-choi bottom rot were compared. Specifically, similarly to Example 1, 2 ml of the pathogenic microbe suspension and 6 ml of each of the suspensions A2, F, and G were placed in a sterilized pot containing cultivation soil separately prepared and mixed thoroughly therein; 20 surface sterilized Pak-choi seeds were added thereto; the disease development was observed in a plant growth chamber under a condition similar to that in Example 1. Results are summarized in FIG. 4.

As apparent from FIG. 4, the suspension A2, F, and G could all suppress the development of the disease. Although the suspension F and G had a control effect weaker than that of the suspension A2 using Coprinus curtus GM-21, the suspension F could reduce the dam age in the early phase of growth to 50% or less, while the suspension G could suppress the development in the early to middle phase of growth.

### Example 5

Coprinus comatus was inoculated in 250 ml of PD liquid medium and incubated at 25° C. for 13 days while being shaken at 110 rpm, and the mixture was homogenized, to give a suspension F2.

The disease-controlling effect of the suspension F2 against Rhizoctonia solani Pak-choi 2 was determined in the following manner:

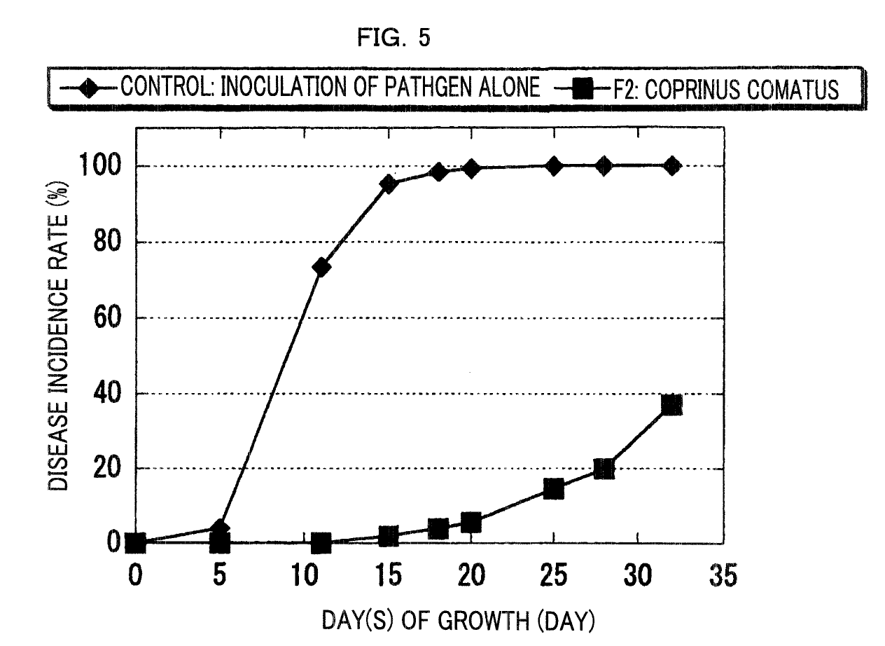

A portion 3 cm×3 cm was cut out from an agar plate of a PD medium having Rhizoctonia solani Pak-choi 2 grown thereon, 50 ml of sterile water was mixed with the agar plate and diluted 10 fold with sterile water, to give a pathogenic microbe suspension; and the control effect of the suspension F2 against Pak-choi bottom rot was evaluated. Specifically, similarly to Example 1, 5 ml of the pathogenic microbe suspension and 5 ml of the suspension F2, and 5 ml of sterile water were placed in a sterilized pot containing cultivation soil separately prepared and mixed thoroughly therein; 20 surface sterilized Pak-choi seeds were added thereto; and the disease state was observed in a plant growth chamber under a condition similar to that in Example 1. Five ml of the suspension F2 was added onto the soil surface additionally on the 15th day after initiation of the test. Results are summarized in FIG. 5.

As apparent from FIG. 5, the Coprinus comatus suspensions, both obtained by stationary and shaking culture, had similar disease-controlling effects. Addition of the suspension F2 obtained by shaking culture during growth was more effective in increasing the disease-controlling effect than addition of the suspension F of Example 4 prepared by stationary culture.

### Example 6

Rhizoctonia solani Pak-choi 2 grown on the agar plate of PD medium and GM-21 grown on the agar plate of PD medium were respectively collected in small portions; and both portions were placed on a separately prepared the agar plate of PD medium, separated by a distance of approximately 5 cm and cultured at 27° C. for 5 days.

Similarly, a melon Fusarium wilt-causing microbe, Fusarium oxysporum f. sp. melonis F0-me-2, and a tomato Fusarium wilt-causing microbe, Fusarium oxysporum f. sp. redicus-lycopersici F0-T-3, were also placed similarly, respectively on the agar plate of PD medium together with GM-21 and incubated at 27° C. for 5 days.

As a result, there was a distinct boundary line formed at the interface between the pathogenic microbe hypha and GM-21 hypha, independent of the pathogenic microbe used. The pathogenic hypha also changed in their shape and color. The results indicate that both the pathogenic microbes was damaged significantly by contact with GM-21.

### Example 7

Fifty ml of sterile water was added to 2 g of GM-21 hypha obtained in a similar manner to Example 1, and a mixture obtained was homogenized, to give a suspension A3.

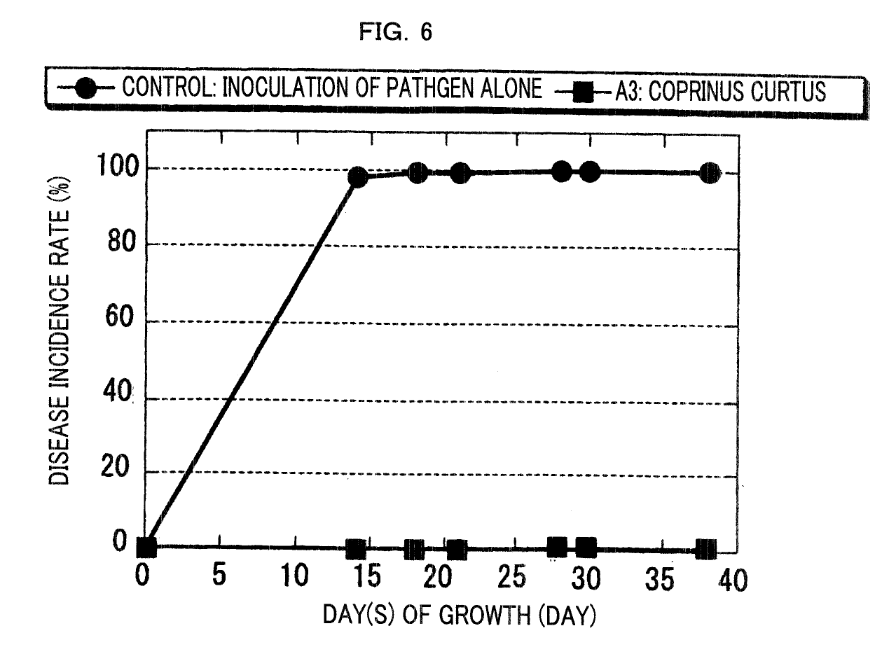

Fifty ml of sterile water was added onto PD agar culture medium having a lettuce bottom rot-causing microbe, Rhizoctonia solani lettuce 2, grown thereon; tile mixture was homogenized and diluted 1000 fold with sterile water, to give a pathogenic microbe suspension; and the control effect of the Coprinus curtus GM-21 suspension A3 on lettuce bottom rot was evaluated. Similarly to Example 1, 2 ml of the pathogenic microbe suspension, 4 ml of the suspensions A3, and 6 ml of sterile water were placed in a sterilized pot containing cultivation soil separately prepared and mixed thoroughly therein; 20 surface sterilized lettuce seeds were added thereto, and the disease development was observed in a plant growth chamber under a condition similar to that in Example 1. Results are summarized in FIG. 6.

As apparent from FIG. 6, the suspension A2 could suppress the development of the pathogenic microbe significantly.

As obvious from the Examples above, with the plant disease control agent and the control method according to the invention, it is possible to control plant diseases caused by fungi. It was also found that the invention was effective not only to a particular plant disease but to a wide range of plant diseases. In particular, the solution containing the fragment of Coprinus curtus GM-21 (Coprinus curtus Kalchbr. ex Thum. GM-21) was effective.

Also, the plant disease control agent according to the invention, which uses an edible mushroom as its raw material, is safer in terms of leaving no residue. The control effect of suspended plant disease control agents was evaluated in the Examples described above, however, the plant disease control agent according to the invention is not limited to such suspensions, and the suspension may be used as a solid material in which the suspension is absorbed or as a controlling agent in another form.

1. A plant disease control agent comprising a fragment of an inky cap mushroom.

2. The plant disease control agent according to claim 1, wherein the inky cap mushroom is selected from the group consisting of the genus Coprinus and the genus Psathyrella.

3. The plant disease control agent according to claim 1, wherein the inky cap mushroom is comprises at least one selected from the group consisting of Coprinus curtus, Coprinus cinereus, Coprinus disseminatus, Coprinus comatus, Coprinus atramentarius, Coprinus radians, Psathyrella multissima, Psathyrella candolleana and Psathyrella velutina.

4. The plant disease control agent according to claim 1, wherein the inky cap mushroom comprises Coprinus curtus GM-21 (NITE BP-37; SEQ ID NO: 1).

5. The plant disease control agent according to claim 1, wherein the plant disease control agent comprises a suspension comprising the fragment of the inky cap mushroom.

6. The plant disease control agent according to claim 5, wherein the plant disease control agent comprises a solid material comprising the suspension and a carrier into which the suspension is absorbed.

7. The plant disease control agent according to claim 1, wherein the plant disease control agent comprises a preparation form selected from the group consisting of a wettable powder, an emulsion, an oil, granules, a powder, a tablet, a capsule and a seed-coating agent.

8. (canceled)

9. (canceled)

10. A method for controlling a plant disease comprising controlling plant diseases by using a plant disease control agent comprising a fragment of an inky cap mushroom.

11. The method for controlling a plant disease according to claim 10, wherein a pathogenic microbe of the plant disease is a fungus.

12. The method for controlling a plant disease according to claim 11, wherein the plant disease fungus is a genus selected from the group consisting of the genus Rhizoctonia and the genus Fusarium.

13. The method for controlling a plant disease according to claim 10, wherein the plant disease is selected from the group consisting of Pak-choi bottom rot, turf leaf rot, melon wilt and tomato wilt.

14. The method for controlling a plant disease according to claim 10, wherein the inky cap mushroom is selected from the group consisting of the genus Coprinus and the genus Psathyrella.

15. The method for controlling a plant disease according to claim 10, wherein the inky cap mushroom is comprises Coprinus curtus GM-21 (NITE BP-37; SEQ ID NO: 1).

16. The method for controlling a plant disease according to claim 10, wherein the plant disease control agent comprises a suspension comprising the fragment of the inky cap mushroom.

17. Coprinus curtus GM-21 (NITE BP-37; SEQ ID NO: 1).


SEQUENCE LISTING (1 sequence)

SEQ ID NO: 1 - DNA, 663 bp, Coprinus curtus GM-21
tttccgtaggtgaacctgcggaaggatcattaacgaataactatggtgttggttgtagctgcctcctcggaggaatgtgcacgcccgccatttttatctttccacctgtgcaccgactgtaggtctggataactctcgcctcacggcagatgcgaggattggcctctgtgcctctcctcgaatttccaggctctacgtcttttacacaccccaatagtatgatatagaatgtagtcaatgggcttcttagcctataaaacactatacaactttcagcaacggatctcttggctctcgcatcgatgaagaacgcagcgaaatgcgataagtaatgtgaattgcagaattcagtgaatcatcgaatctttgaacgcaccttgcgctccttggtattccgaggagcatgcctgtttgagtgtcattaaattctcaacctcgccagttttctgaactgcgtcgaggcttggattgtgggggtttgtgcaggctgcctcagcgtggtctgctcccctgaaatgcattagcgagttcatactgagctccgtctatcggtgtgataattatctacgccgttagtcgagttcagacttgcttctaaccgtccgcaaggacaactcttgacaatttgacctcaaatcaggtaggactacccgctgaacttaa